(19) United States
(12) Patent Application Publication
(10) Pub. No.: US 2025/0248846 A1
(43) Pub. Date: Aug. 7, 2025

(54) OPHTALMOLOGICAL INTERPOSITION IMPLANT WITH ENGAGEMENT PROTUSION

(71) Applicant: CILIATECH, Chavanod (FR)

(72) Inventors: Olivier BENOIT, Epagny Metz-Tessy (FR); Philippe SOURDILLE, GUERANDE (FR)

(21) Appl. No.: 18/857,027

(22) PCT Filed: Apr. 14, 2023
(86) PCT No.: PCT/FR2023/050543

§ 371 Date: Oct. 15, 2024

(30) Foreign Application Priority Data
Apr. 15, 2022 (FR) FR2203542

(51) Int. Cl. A61F 9/007

(52) U.S. Cl. CPC A61F 9/00781; A61F 2250/0036

(57) ABSTRACT

An ophthalmological implant (20) for permanent interposition between the sclera and the uveal tissue incudes a uvea-compatible body (22), the body having three dimensions in space, namely a length and a width which are perpendicular to each other and to a thickness. The body of the implant has a first edge (24), referred to as the front edge, which is intended to be oriented in the direction of the front chamber of an eye in contact with the base of the ciliary muscle, and a second edge (26), referred to as the rear edge, which is opposite the front edge (24) relative to the body (22). The front edge has, in a projection view in the plane containing the width and the length, at least one protrusion (32) oriented toward the outside of the body (22) to allow at least local contact of the at least one protrusion on the base of the ciliary muscle.

The invention relates to an interpositional ophthalmological implant intended to collect the aqueous humor from the anterior chamber and convey it to the supra-ciliary and supra-choroidal space in order durably to lower intraocular pressure (IOP).

Intraocular pressure is the result of an equilibrium reached between secretion of aqueous humor by the ciliary body and its flow through the corneoscleral trabecular meshwork via the Schlemm's canal and its clearance to the aqueous veins and general circulation. A fraction representing 5 to 30% depending on age of this flow flows directly through the ciliary trabecula between the sclera and the ciliary body, and this is what is referred to as the uveo-scleral flow. The longitudinal fibers of the ciliary muscle, particularly during accommodation, play a role in tensioning the trabecular meshwork, facilitating the uveo-scleral flow of the aqueous humor. It is also via these longitudinal fibers of the ciliary muscle that the uveo-scleral flow flows.

In glaucoma, the flow of aqueous humor is reduced at the level of the corneo-scleral trabecula, and this in most cases leads to an increase in intraocular pressure (IOP). Lowering this IOP is therefore the key factor in the medical and/or surgical treatment of glaucoma. Surgical treatment relies on two options: reducing the production of aqueous humor produced by the ciliary body (cyclo-weakening) or increasing the flow of aqueous humor by diverting it. This diversion is performed in different ways:
by making the anterior chamber communicate directly with the suprachoroidal space (cyclodialysis and technical derivatives thereof), but the effect obtained is usually transient and insufficient. By disinserting the insertion of the ciliary muscle in the scleral spur this implantation technique eliminates the physiological mechanism for the uveo-scleral flow in the surgical zone. In addition, post-operative fibrosis extends beyond this zone. It is also known that the part of the implant situated in the anterior chamber may, even if only intermittently, touch the corneal endothelium, thereby leading to a significant risk of evolving corneal edema,
by incising the trabecula, working from the anterior chamber, as far as the Schlemm's canal in order to short-circuit the trabecular obstacle. This intervention may be supplemented by the insertion into the canal of a stent open toward the anterior chamber in order to maintain permanent direct access for the aqueous humor to the canal. Once again, the results are often partial and temporary, and do not obviate the need for continuation or resumption of medical treatment.

Filtration surgery continues to be a treatment of choice and seeks to divert the aqueous humor under the conjunctiva in order to achieve the necessary drop in pressure. It may create a full-thickness permanent hole in the trabecula under a scleral flap: this is known as a trabeculectomy. One variant also leaves the internal part of the trabecula in place, and this is what is referred to as non-penetrating trabecular surgery (deep sclerotomy, visco-canalostomy).

However, filtering surgery runs into complications connected with the insufficiency of the filtering by fibrosis of the filtration bubble (the filtration bubble is present between the sclera and the conjunctiva, which is raised) or, conversely, excessive filtering, which may be linked to the use in surgery of mytomycin C.

There are moreover also known from the prior art numerous attempts to normalize the IOP thanks to implants, either used on their own or in conjunction with the surgical procedures cited below. Now, these implants all deform the anatomy of the eye and/or divert/modify the natural flow channels to a greater or lesser degree. Furthermore, these implants are intended to match the dimensions of a standard eye; they do not adapt to the anatomical variations present in nature.

It is known in particular from the document WO 2016/156727 A1 to implant an intraocular implant making it possible to increase and to make permanent the hypotensive effect of the physiological uveo-scleral flow by positioning between the sclera and the ciliary body an implant that does not alter the anatomical structures, with or without added filtration or other intervention.

The implant in the document WO 2016/156727 A1 comprises a body comprising an anterior edge intended to be situated as close as possible to the uveal trabecula when the implant is between the sclera and the ciliary body. This anterior edge is concave and forms an edge allowing linear and continuous contact between the anterior edge and the ciliary muscle and its tendon at the level of its insertion in the scleral spur. This geometry in particular enables intimate contact with the flow zone of the uveal trabecula.

A drawback identified with this type of interpositional ophthalmological implant having a linear and continuous contact with the flow zone of the uveal trabecula, like that in the document WO 2016/156727 A1, is that the ophthalmological implant blocks some of the flow of aqueous humor because of the narrow contact between the tissues and the ophthalmological implant.

Another drawback with this type of ophthalmological implant is that they do not adapt to the anatomical variability that may be encountered in patients. In particular, it has been observed that the diameter of the anterior chamber of the eye in patients can vary between 10.75 mm and 13.75 mm. Thus the performance of the ophthalmological implant can depend on the anatomy of the patient.

Thus there exists a need for an ophthalmological implant free of the drawbacks mentioned hereinabove. In particular, there exists a need for an ophthalmological implant favoring to a greater degree the flow of the aqueous humor from the uveal trabecula whilst preserving correct anterior positioning and adapting to different anatomies of patients.

To this end the invention proposes an ophthalmological implant for permanent disposition between the sclera and uveal tissue, characterized in that the implant comprises a uveo-compatible body, consisting of a single part, the body having three dimensions in space, namely a length and a width that are perpendicular to one another and a thickness, the body of the implant comprising a first or anterior edge intended to be oriented in the direction of the anterior chamber of an eye in contact with the root of the ciliary muscle and a second or posterior edge opposite the anterior edge relative to the body. Given the required position of the implant the anterior edge of the implant may be in contact only with the posterior wall of the root of the ciliary muscle. The anterior wall of the root of the ciliary muscle is located in the anterior chamber and constitutes one of the limits thereof. Use of the term “in contact with the root of the ciliary muscle” as used in the present document must be understood as meaning “in contact with the posterior wall of the root of the ciliary muscle”.

As seen in a view in projection in the plane containing the width and the length, the anterior edge has at least one protuberance oriented toward the exterior of the body to enable at least local contact of said at least one protuberance on the root of the ciliary muscle.

A geometry of the implant inducing local contact between the anterior edge and the root of the ciliary muscle enables space to be freed up between the body of the implant and the root of the ciliary muscle favoring the release of the aqueous humor from the uveal trabecula and flow thereof toward the ciliary body and the sclera.

Furthermore, whether single or multiple, the local contact enables the anterior edge to adapt to different patient anatomies. In fact, it is easier for the implant to be positioned against the root of the ciliary muscle despite the different diameters of the anterior chamber.

By “local” contact is meant that solely a portion of the anterior edge is intended to be in contact with the root of the ciliary muscle. The anterior edge is therefore shaped so that it has no contact with the root of the ciliary muscle over the entirety of the anterior edge.

In one embodiment of the implant the anterior edge comprises a plurality of protuberances oriented toward the exterior of the body, said protuberances conjointly forming a discontinuous or local contact edge on the root of the ciliary muscle.

In one embodiment of the implant the contact edge defines a contact profile of the anterior edge on the root of the ciliary muscle, this contact profile extending along a concave trajectory.

In one embodiment of the implant the contact profile of the contact edge extends along at least one circular trajectory.

In one embodiment of the implant the contact profile of the contact edge comprises at least one profile portion extending along a circular trajectory.

In one embodiment of the implant the contact profile of the contact edge comprises at least two profile portions extending along circular trajectories having different radii of curvature.

In one embodiment of the implant the contact profile of the contact edge comprises at least one first profile portion extending along a first circular trajectory having a first radius of curvature and at least one second profile portion extending along a second circular trajectory having a second radius of curvature greater than the first radius of curvature to enable the contact edge to come into iris contact with the root of the ciliary muscle having a plurality of possible anatomical radii.

In one embodiment of the implant the first profile portion is formed at the level of a central zone of the contact edge, the second profile portion being disposed at the level of a peripheral zone of the contact edge.

In one embodiment of the implant the second profile portion is disposed on either side of the first profile portion along the contact edge.

In one embodiment of the implant at least one radius of curvature of the circular trajectory is greater than or equal to 4.5 mm and less than or equal to 6 mm.

In one embodiment of the implant at least one radius of curvature of the circular trajectory is greater than 6 mm and less than or equal to 7.5 mm.

In one embodiment of the implant said at least one protuberance is configured to allow at least one localized contact on the root of the ciliary muscle.

In one embodiment of the implant said at least one protuberance is configured to enable at least one linear contact on the root of the ciliary muscle.

In one embodiment of the implant each protuberance comprises a contact edge portion, each of said contact edge ports extending along the concave trajectory.

In one embodiment of the implant the thickness of the anterior edge is thinned relative to the thickness of the body.

In one embodiment of the implant the anterior edge comprises an upper portion and a lower portion, the thickness of the anterior edge being thinned at the level of the upper portion.

In one embodiment of the implant the body comprises a local thinning of thickness situated set back from the anterior edge and configured to enable elastic deformation of the anterior edge by the action of a force tending to press the anterior edge against a bearing surface, for example the root of the ciliary muscle.

In one embodiment of the implant the anterior edge comprises at least one needle extending toward the exterior of the body and projecting beyond said at least one protuberance in such a manner as to be able to pass through a wall of the root of the ciliary muscle when said at least one protuberance is in contact with the root of the ciliary muscle.

In one embodiment of the implant the needle comprises a perforation distal point configured to perforate a wall of the root of the ciliary muscle without an opening being formed in said wall beforehand.

In one embodiment of the implant said at least one needle comprises an aqueous humor collecting channel extending from one end of said at least one needle to the body of the implant.

In one embodiment of the implant the body forms at least one aqueous humor collecting recess opening at the level of the anterior edge.

In one embodiment of the implant said at least one recess is formed between two protuberances.

In one embodiment of the implant the body of the implant is elastically deformable in such a manner as to be able to be folded without permanent deformation in order to be manipulated by a micro-instrument or injected using an ophthalmological injection system.

In one embodiment of the implant the body of the implant is configured to have at least one radius of curvature of the circular trajectory that is greater than or equal to 4.5 mm and less than or equal to 6 mm when the implant is disposed in a deformed state corresponding to a disposition of the body of the implant between the sclera and uveal tissue.

In one embodiment of the implant the body of the implant is configured to have at least one radius of curvature of the circular trajectory that is greater than 6 mm and less than or equal to 7.5 mm when the implant is disposed in said deformed state corresponding to a disposition of the body of the implant between the sclera and the uveal tissue.

In one embodiment of the implant the body of the implant has in a deformed state in which it can be used as an interpositional ophthalmological implant between the sclera and the uveal tissue a concave curvature in a direction perpendicular to a plane defined by the two dimensions of the implant that are perpendicular to the thickness.

In one embodiment of the implant the body of the implant is made of a material that has a Young's modulus between 30 and 60 kg/cm² or between 30,000 and 2,500,000 kg/cm².

In one embodiment of the implant the body of the implant is not elastically deformable and has a permanent curvature in a direction perpendicular to a plane defined by the two dimensions of the implant that are perpendicular to the thickness.

In one embodiment of the implant the body of the implant comprises at least one material that is chosen from the following materials: PTFE, polysiloxane, hydrophilic or hydrophobic acrylate hydrogels.

In one embodiment of the implant the body of the implant has two opposite large faces separated from one another by the thickness of the body.

In one embodiment of the implant the two opposite large faces comprise an upper face and a lower face separated by the thickness, the body of the implant having one of the following conformations in a direction perpendicular to a plane defined by the two dimensions of the implant that are perpendicular to the thickness:
the upper face is plane and the lower face is concave,
the upper face is plane and the lower face is convex,
the upper and lower faces are convex,
the upper and lower faces are plane.

In one embodiment of the implant the body is hollow and forms a central cavity between the upper and lower faces. The central cavity may be open to the outside, for example at the level of one or more of the lateral edges and the posterior edge. This central cavity is completely or partially empty of material, in particular over the anterior-posterior distance.

In one embodiment of the implant the body is formed by a plate folded on itself in such a manner as to form the central cavity between at least two facing portions of that plate. The body can therefore be formed by a mainly two-dimensional element, here a plate, the arrangement of which, here by folding, enables a three-dimensional body to be formed the thickness of which is greater than the combined thicknesses of the two facing portions of the plate.

In one embodiment of the implant the body of the implant comprises at least one material chosen from the following materials: polypropylene, plexiglass, titanium, stainless steel, Nitinol.

In one embodiment of the implant the body of the implant is pierced by orifices passing through its thickness and/or the body of the implant comprises on at least one of its two opposite large faces a relief that is adapted to favor flow of the aqueous humor across said at least one large face.

In one embodiment of the implant the relief on said at least one large face of the body takes the form of recesses formed on said at least one large face or a roughness conferred thereon.

In one embodiment of the implant the thickness (e) of the body of the implant is between 50 and 1000 μm.

In one embodiment of the implant the body of the implant has properties of releasing one or more substances.

In one embodiment of the implant the posterior edge is at a distance of less than 1.0 mm from the anterior edge.

The invention may optionally concern a first method of inserting a permanent interpositional ophthalmological implant between the sclera and the uveal tissue in which the implant may conform to the implant described hereinabove, the method being carried out after conventional intraocular surgical intervention has been performed on the eye of the patient and one of more scleral flaps in said eye of the patient have been cut, the method comprising the following steps:
raising one or more scleral flaps;
making at least one incision extending as far as the ciliary body;
inserting the implant between the sclera and the ciliary body;
positioning the implant as close as possible to the trabecula in such a manner as to position said at least one protuberance in contact with the root of the ciliary muscle.

The invention may optionally concern a second method of inserting a permanent interpositional ophthalmological implant between the sclera and the uveal tissue in which the implant may conform to the implant described hereinabove, the method comprising the following steps:
making at least one incision extending as far as the ciliary body;
inserting the implant between the sclera and the ciliary body through said at least one incision;
positioning the implant as close as possible to the trabecula in such a manner as to position said at least one protuberance in contact with the root of the ciliary muscle.

In one embodiment of the first or second insertion method said at least one incision is concentric relative to the limbus.

In one embodiment of the first or second insertion method said at least one incision is perpendicular relative to the limbus.

In one embodiment of the first or second insertion method the anterior edge comprises at least one needle, said method comprising the production of at least one opening between the anterior chamber and the supraciliary space, the step of positioning the implant comprising the insertion of said at least one needle in said at least one opening between the anterior chamber and the supraciliary space until said at least one protuberance is in contact with the root of the ciliary muscle.

In one embodiment of the first or second insertion method the number of openings made between the anterior chamber and the supraciliary space is at least equal to the number of perforating needles of the anterior edge.

In one embodiment of the first or second insertion method the anterior edge comprises at least one needle having a perforation distal point, the step of positioning the implant comprising perforation of the root of the ciliary muscle by the perforation distal point until said at least one protuberance is in contact with the root of the ciliary muscle.

In one embodiment of the first or second insertion method the latter further comprises, before insertion of the implant, injection of a visco-elastic substance between the sclera and the ciliary body through said at least one incision so as to separate these two tissues.

### BRIEF DESCRIPTION OF THE DRAWINGS

The appended drawings depict the invention:

FIG. 1 represents a highly-schematic general view showing from the front one possible embodiment of an implant according to the invention positioned around the cornea of an eye.

FIG. 2 represents a detailed view of an anterior edge of an implant according to the invention comprising a plurality of protuberances enabling contact with the root of the ciliary muscle of the eye.

FIG. 3 represents a detailed view of an anterior edge of an implant according to the invention comprising a plurality of protuberances forming contact edge portions extending along circular trajectories having different radii of curvature.

FIG. 4 represents a side view of an implant according to the invention in which the anterior edge is thinner than the body of the implant.

FIG. 5 represents a side view of an implant according to the invention in which the anterior edge is thinner than the body of implant, the anterior edge also forming a local thinning on a lower or upper face of the implant.

FIG. 6 represents a first embodiment of the anterior edge of an implant according to the invention comprising a protuberance.

FIG. 7 represents a second embodiment of the anterior edge of an implant according to the invention comprising a protuberance.

FIG. 8 represents a third embodiment of the anterior edge of an implant according to the invention comprising two protuberances.

FIG. 9 represents a fourth embodiment of the anterior edge of an implant according to the invention comprising two protuberances.

FIG. 10 represents a fifth embodiment of the anterior edge of an implant according to the invention comprising two protuberances.

FIG. 11 represents a sixth embodiment of the anterior edge of an implant according to the invention comprising two protuberances.

FIG. 12 represents a seventh embodiment of the anterior edge of an implant according to the invention comprising two protuberances.

FIG. 13 represents an eighth embodiment of the anterior edge of an implant according to the invention comprising two protuberances.

FIG. 14 represents a ninth embodiment of the anterior edge of an implant according to the invention comprising two protuberances forming contact edge portions extending along circular trajectories with different radii of curvature.

FIG. 15 represents highly schematically a first method of inserting an implant.

FIG. 16 represents highly schematically a second method of inserting an implant.

FIG. 17 represents a schematic view in position in a human eye of possible simplified general shape of an implant according to the invention disposed between the sclera and the ciliary body.

FIG. 18 represents a more detailed view to a larger scale of the structure of the iridocorneal angle without the FIG. 17 implant.

FIG. 19 represents a view from above of an implant comprising a plurality of protrusions and a first embodiment of a needle intended to be inserted in the anterior chamber of an eye.

FIG. 20 represents an implant comprising a second embodiment of a needle.

FIG. 21 represents an implant comprising a third embodiment of a needle.

FIG. 22 represents an implant comprising a fourth embodiment of a needle.

FIG. 23 represents a side view of an implant in which the needle is thinner than the body of the implant.

FIG. 24 represents a view from above of an implant in which the width of the needle decreases from a proximal end attached to the body to a distal end to facilitate the insertion of the needle in the anterior chamber of the eye.

FIG. 25 represents a side view of an implant in which the thickness of the needle is greater than the thickness of the body of the implant.

FIG. 26 represents an implant comprising two needles intended to be inserted in the anterior chamber of an eye.

FIG. 27 represents in a highly-schematic manner the fitting of an implant by a third method.

FIG. 28 represents a side view of a needle having a perforation distal point having a beveled edge to enable perforation of the tissues of the root of the ciliary muscle.

FIG. 29 represents a perspective view of a first example of a hollow implant the upper and lower faces of which are separated by a central cavity.

FIG. 30 represents a perspective view of a second example of a hollow implant the upper and lower faces of which are separated by a central cavity.

FIG. 31 represents a side view of the implant from FIG. 30.

FIG. 32 represents a third example of a hollow implant the upper and lower faces of which are separated by a central cavity and that includes a needle with a beveled perforation distal point.

FIG. 33 represents a side view of the implant from FIG. 32.

### DESCRIPTION OF EMBODIMENT(S)

The concept of the invention is described more completely hereinafter with reference to the appended drawings in which embodiments of the concept of the invention are shown. In the drawings the sizes and the relative sizes of elements may be exaggerated for purposes of clarity. Similar numbers refer to similar elements in all the drawings. However, this concept of the invention may be reduced to practise in numerous different forms and should not be interpreted as being limited to the embodiments disclosed here. Rather than that, these embodiments are proposed so that this description is complete and communicates to persons skilled in the art the extent of the concept of the invention.

A reference throughout the specification to “an embodiment” means that their particular functionality, structure or feature described with reference to one embodiment is included in at least one embodiment of the present invention. Thus the appearance of the expression “in one embodiment” at various places throughout the specification does not necessarily refer to the same embodiment. Furthermore, the particular functionalities, structures or features may be combined in any appropriate manner in one or more embodiments. Moreover, the term “comprising” does not exclude other elements or steps.

The present invention is depicted with the aid of the figures, which show an interpositional ophthalmological implant that is intended to collect aqueous humor from the anterior chamber to the supra-choroidal space to reduce the intra-ocular pressure (IOP) durably.

For reasons of clarity in the present disclosure the interpositional ophthalmological implant may be referred by the term “implant” hereinafter.

The implant comprises a uveo-compatible body, i.e. is made of at least one material that is known for its uveo-compatibility properties. Such properties impart to the body a very low adhesion to the ocular tissues. In other words, said at least one material is not liable to degrade the overlying and underlying structures following the body coming into contact with those structures and repeated movements thereof over time.

The body of the implant has three dimensions in space: a thickness and a length and a width that are perpendicular to the thickness. The ratio between the length and the width may be less than, equal to or greater than 1. For example, the implant may have for dimensions (width, length, thickness) 4*5*0.1 mm, or 3*7*0.8 mm, or 6*3*0.6 mm. Furthermore, the thickness of the implant may vary between the anterior edge and the posterior edge, for example between 0.15 mm at the anterior edge and 0.7 mm at the posterior edge. This variation may be gradual, random, over the entirety of the anterior-posterior travel, and/or local.

The implant is preferably made as one part in the sense that it is formed as a monobloc single piece. The implant is therefore not an assembly of a plurality of parts fixed to one another to form the body.

The body of the implant is preferably thin so that, once placed between the sclera and the uveal tissue, it does not deform the overlying and underlying tissues unacceptably. An acceptable deformation of the tissues is a deformation that does not degrade the function or functions of one and/or the other of those tissues. The thickness of the body 22 of the implant 20 is preferably between 50 and 1000 μm.

The body of the implant comprises two opposite edges that are at a distance from one another in one of the two directions perpendicular to the thickness. The two opposite edges comprise an anterior edge and a posterior edge opposite the anterior edge relative to the body.

The anterior edge is intended to be oriented in the direction of or facing the anterior chamber of an eye and placed in contact with the posterior wall of the root of the ciliary muscle.

In FIG. 1 an eye 10 is represented highly schematically from the front by the cornea 12, the pupil 14 at the center and the root of the ciliary muscle 118. When the root of the ciliary muscle 18 is mentioned in the present text that means the posterior wall of the root of the ciliary muscle.

An implant 20 comprises a body 22 having an anterior edge 24 and a posterior edge 26. Lateral edges 28 and 30 extend either side of the body between the anterior edge 24 and the posterior edge 26. The geometry of the implant in FIG. 1 is schematic. The objective of FIG. 1 is to define the position of the implant 20 relative to the root of the ciliary muscle 118 and to identify the anterior edge 24, the posterior edge 26 and the lateral edges 28, 30. The geometrical characteristics of the implant 20 are described in detail below.

The posterior edge 26 is preferably convex. A convex edge traces out a curve departing from the body 22 of the implant 20 or conversely a concave edge traces out a re-entrant curve or a curve approaching the body 22 of the implant 20. Alternatively, the posterior edge 26 may be rectilinear or concave.

In one possible embodiment the convexity of the posterior edge 26 is extended by the lateral edges 28, 30, i.e. with no angle formed between the lateral edges 28, 30 and the posterior edge 26.

Generally speaking, whatever the shape of the implant, the posterior edge 26 must be sufficiently far from the anterior edge to procure an effective separation effect. In practise the posterior edge is preferably at a distance of at least 1.0 mm from the anterior edge.

Generally speaking, whatever the shape of the implant, the length of the posterior edge 26 does not exceed 50% of the length of the anterior edge.

The lateral edges 28, 30 are symmetrical with respect to one another although, in a variant that is not represented, they may be asymmetrical relative one another.

The lateral edges 28, 30 may be arranged in a radial manner (lateral edges converging at an imaginary point situated in front of the anterior edge), in a flared manner (lateral edges converging at an imaginary point situated in front of the posterior edge) or parallel to one another.

The body 22 of the implant 20 has two opposite large faces separated from one another by the thickness of the body 22. The two opposite large faces comprise an upper face and a lower face separated by the thickness. The upper face is intended to be in contact with the sclera. The lower face is intended to be in contact with the ciliary body.

The body 22 of the implant 20 may have any one of the following shapes in a direction perpendicular to a plane defined by the two dimensions of the implant that are perpendicular to the thickness:
the upper face is plane and the lower face is concave,
the upper face is plane and the lower face is convex,
the upper and lower faces are convex,
the upper and lower faces are plane.

In one embodiment the body 22 may be hollow. Thus the body 22 can form a central cavity between the upper and lower faces. The central cavity may be open to the outside of the implant, for example at the level of one or more of the lateral edges and the posterior edge. This central cavity is wholly or partially empty of material, in particular along the anterior-posterior distance.

In one embodiment of the implant the body is formed by a plate folded on itself in such a manner as to form the central cavity between at least two facing portions of that plate. The body can therefore be formed by a mainly two-dimensional element, here a plate, the arrangement of which, here by folding, enables a three-dimensional body to be formed the thickness of which is greater than the combined thicknesses of the two facing portions of the facing plate. Some or all of the walls of the implant 20, namely the upper and lower faces, the lateral edges and the anterior and posterior edges may be apertured. In other words, these walls may be produced in the form of a meshing causing an alternation of material and holes to appear.

This meshing may be obtained by removal of material or by weaving filaments of material. The implant 20 may be produced in the form of a plurality of woven and mainly two-dimensional walls forming a three-dimensional envelope enclosing the central cavity. An embodiment of this kind using apertured walls makes it possible to favor the collection and the flow of the aqueous humor. The implant 20 may comprise a combination of apertured walls and solid walls.

The anterior edge 24 includes at least one protuberance 32 oriented toward the exterior of the body 22. Said at least one protuberance 32 projects out of the body 20 as seen in a view in projection in the plane containing the width and the length, as can be seen in FIG. 1. This protuberance 32 enables at least local contact or said at least one protuberance on the root of the ciliary muscle.

By “local” contact is meant the fact that only a portion of the anterior edge 24 is intended to be in contact with the root of the ciliary muscle. The anterior edge 24 is therefore shaped so that there is no contact with the root of the ciliary muscle over the entirety of the anterior edge 24.

The anterior edge 24 may comprise a single protuberance 32 of the material so as to simplify the geometry of the anterior edge 24 at the same time as maximizing the collection of aqueous humor. Said protuberance 32 is preferably disposed at the level of a central part of the anterior edge 24 to enable better positioning of the implant 20 on the root of the ciliary muscle.

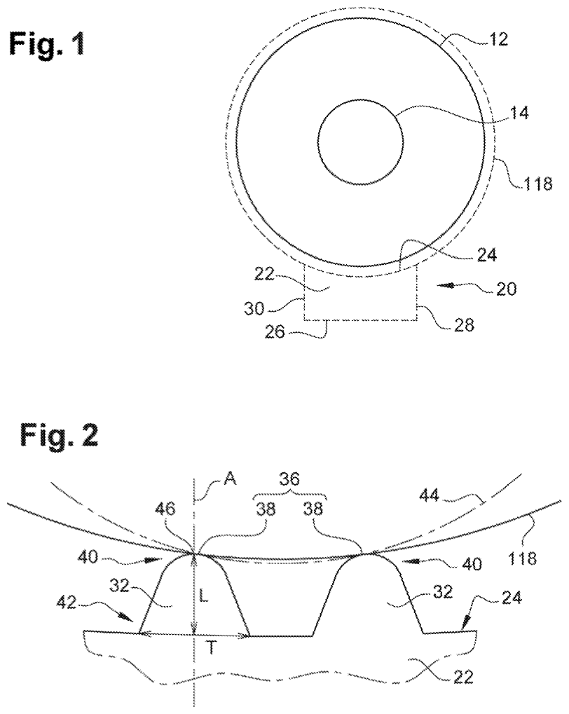

Referring to FIG. 2, the anterior edge 24 comprises a contact edge 36 that forms the contact zone between the anterior edge 24 and the root of the ciliary muscle 118. Each protuberance 32 of said at least one protuberance 32 comprises a contact edge portion 38 at the level of a distal end 40. If the anterior edge comprises a plurality of protuberances 32 the contact edge 36 is formed by the plurality of contact edge portions 38 carried by the protuberances 32.

In the case of a plurality of protuberances 32 the latter conjointly form a discontinuous or local contact edge 36. In other words, the contact between the anterior edge 24 and the root of the ciliary muscle is discontinuous because it is formed by a plurality of contact edge portions 38. Furthermore, the contact may be local in that it is not achieved by the entirety of the anterior edge 24 but by only a part of that anterior edge 24. The contact edge 36 preferably enables discontinuous and local contact in that the contact is achieved over a plurality of localized contact zones.

Increasing the number of contact edge portions 38 enables improved positioning and stability of the implant 20 against the root of the ciliary muscle 118, which improves the collection of aqueous humor.

Said at least one protuberance 32 may be configured to enable at least one localized contact on the root of the ciliary muscle. It should be noted that the tissues of the ciliary muscle are flexible and that the concept of localized contact remains theoretical. By deforming, the tissues will generate contact over a contact zone around this theoretical contact point. Thus there is meant by “localized” contact a contact zone localized at the level of a portion of the protuberance 32 having a curved or convex profile. Thus the contact edge portion 38 of said at least one protuberance 32 may have a convex profile enabling theoretical localized contact with a spherical surface such as the root of the ciliary muscle.

The convex profile of the contact edge portion 38 may be produced by a curved edge or by a plurality of rectilinear edges along a curved profile.

Said at least one protuberance 32 may be configured to enable at least one linear contact on the root of the ciliary muscle 118. The contact edge portion 38 of said at least one protuberance 32 may therefore have a rectilinear or concave profile enabling linear contact with a spherical surface such as the root of the ciliary muscle.

The concave profile of the contact edge portion 38 may be produced by a curved edge or by a plurality of rectilinear edges along a curved profile.

Said at least one protuberance 32 extends along a longitudinal axis A passing through the contact edge portion 38.

Said at least one protuberance 32 forms a projection or a step relative to the body 22 enabling local contact with a convex surface. This projection or step is preferably separated from the rest of the body 22 by a distance at least equal to 0.05 mm. This step value or longitudinal dimension L is measured along the longitudinal axis A between the distal end 40 and the proximal end 42 of said at least one protuberance 32.

Said at least one protuberance 32 may be of elongate shape. Said at least one protuberance 32 has, as seen in a view in projection in the plane containing the width and the length, a transverse dimension T at the level of its proximal end 42 in a direction perpendicular to the longitudinal axis A less than its longitudinal dimension L.

Alternatively, said at least one protuberance 32 may be of flattened shape, i.e. have, as seen in projection in the plane containing the width and the length, a transverse dimension T at the level of its proximal end 42 in a direction perpendicular to the longitudinal axis A greater than its longitudinal dimension L.

The lateral walls said at least one protuberance 32 may be rectilinear, curved or a combination of rectilinear and curved walls.

Said at least one protuberance 32 may also include variations of width, i.e. of transverse dimension T, or a geometry enabling said at least one protuberance 32 to deform elastically, i.e. without permanent deformation, along its longitudinal axis A. In particular, this geometry favoring longitudinal elastic deformation may be an S shape or spring shape.

More generally, the contact edge 36 defines a contact profile 44 of the anterior edge on the root of the ciliary muscle. When the anterior edge 24 comprises a plurality of protuberances 32 each contact edge portion 38 extends along that contact profile 44.

The contact profile 44 preferably extends along a concave trajectory so as to improve the contact between the anterior edge 24 and the convex surface that traces out the root of the ciliary muscle. The concavity of the trajectory of the contact profile 44 is considered facing the implant 20. Thus there is meant by a concave trajectory the fact that the concave edge 36 extending along that profile traces out a concave contact edge capable of adapting optimally to a convex surface such as the root of the ciliary muscle.

The contact profile 44 may in particular extend along a circular trajectory in such a manner as to facilitate the manufacture of the implant, in particular when it is made by machining. Thus each contact edge portion 38 extends along a portion of this circular trajectory.

The contact profile 44 may also extend along different trajectories. To this end the contact profile 44 comprises a plurality of contact profile portions 46 conjointly forming the contact profile 44. A contact profile portion 46 may be carried by one or more contact edge portions 38, i.e. protuberances 32.

To obtain a good compromise between positioning the implant 20 and improving the collection of aqueous humor it is possible to form groups, for example pairs, of protuberances 32 extending along the same trajectory. The anterior edge 24 can thus include a first group of protuberances 32 extending along a first trajectory capable of adapting optimally to a first ciliary muscle geometry and a second group of protuberances extending along a second trajectory capable of adapting optimally to a second ciliary muscle geometry. These groups or pairs of protuberances 32 are preferably distributed along the anterior edge 24 to improve the stability of the contact with the root of the ciliary muscle.

These contact profile portions 46 can in particular extend along different trajectories to obtain a geometry of the anterior edge 24 capable of adapting to multiple surface shapes. Thus it is possible for one or more contact profile portions 46 to extend along a first trajectory and one or more contact profile portions 46 to extend along a second trajectory. The trajectory may be different shapes, at different positions, with different orientations or a combination of the above different attributes. The contact profile 44 can therefore extend successively along rectilinear and/or concave trajectories.

The contact profile 44 may in particular extend along at least one circular trajectory. Thus the contact profile 44 may extend along a plurality of circular trajectories. In other words, the contact edge portions 38 of the protuberances 32 may extend along different circular trajectories.

In one advantageous embodiment the contact profile 44 may extend along circular trajectories having different radii of curvature. In other words, the contact edge portions 38 of the protuberances 32 may extend along circles with different diameters. It is therefore possible to have protuberances 32 the conformation of which, in particular the shape and the orientation, adapt to convex surfaces with different radii. Consequently, the anterior edge 24 is able to adapt to different anatomies, in particular to eyes having different anterior chamber diameters.

These circular trajectories have a center preferably situated in the same direction transverse to the anterior edge 24 as seen in a view in projection in the plane containing the width and the length.

In one advantageous embodiment the anterior edge 24 comprises groups of protuberances 32 extending along circular trajectories having different radii of curvature. In other words the contact profile 44 of the contact edge 36 comprises at least one first profile portion extending along a first circular trajectory having a first radius of curvature and at least one second profile portion extending along a second circular trajectory having a second radius of curvature greater than the first radius of curvature to enable the contact edge to come into contact with ciliary muscle roots having a plurality of possible anatomical radii.

The first profile portion is formed at the level of a central zone of the contact edge, the second profile portion being disposed at the level of a peripheral zone of the contact edge. Thus the second profile portion is disposed either side of the first profile portion along the contact edge when a plurality of contact edge portions, and therefore of protuberances 32, extends along this second trajectory.

It has been determined that the diameter of the anterior chamber of the eye can vary between 10.75 mm and 13.75 mm. Thus the anterior edge 24 is conformed so that at least one radius of curvature of the circular trajectory is greater than or equal to 4.5 mm and less than or equal to 6 mm. Additionally or alternatively the anterior edge 24 is conformed so that at least one radius of curvature of the circular trajectory is greater than 6 mm and less than or equal to 7.5 mm.

Thus the anterior edge 24 comprises at least one group of protuberances 32 extending along a circular trajectory having a radius of curvature greater than or equal to 4.5 mm and less than or equal to 6 mm and at least one group of protuberances 32 extending along a circular trajectory having a radius of curvature greater than 6 mm and less than or equal to 7.5 mm.

In a first configuration the body 22 of the implant 20 is not elastically deformable and has a permanent curvature in a direction perpendicular to a plane defined by two dimensions of the implant 20 that are perpendicular to the thickness. In this embodiment the implant 20 is therefore not elastically deformable and can have one or more anatomical curvatures. In other words the implant 20 can be inserted without elastic deformation into its location against the root of the ciliary muscle.

In a second configuration the body 22 of the implant 20 is elastically deformable in such a manner as to be folded without permanent deformation so as to be manipulated using a micro-instrument or injected using an ophthalmological injection system. When a body 22 of this kind is no longer subjected to the folding force it resumes its original and deformed (rest) position. In this embodiment the implant 20 is therefore an elastically deformable implant and can have one or more anatomical curvatures. In other words the implant 20 is intended only to be placed in a deformed state for its insertion through the tissues.

In a third configuration the body 22 of the implant 20 is elastically deformable in such a manner as to be folded without permanent deformation so as to be manipulated using a micro-instrument or injected using an ophthalmological injection system. When a body 22 of this kind is no longer subjected to the folding force it remains positioned in situ in a deformed state corresponding to the anatomical space between the sclera and the uveal tissue. In other words the implant 20 is intended to be placed against the root of the ciliary muscle in a deformed state for its insertion through the tissues as well as during its use.

When positioned between the sclera and the uveal body the body 22 of the implant 20 is configured to have at least one radius of curvature of the circular trajectory that is greater than or equal to 4.5 mm and less than or equal to 6 mm. Additionally or alternatively the body 22 of the implant 20 is configured to have at least one radius of curvature of the circular trajectory that is greater than 6 mm and less than or equal to 7.5 mm. These ranges of radii of curvature values concern each of the three configurations mentioned hereinabove. In the third configuration these ranges concern the body of the implant 20 when it is in the deformed state and positioned between the sclera and the uveal body. Thus the body of the implant 20 is adapted to the diversity of anatomies of the eye of patients when it is positioned between the sclera and the uveal body whatever the configuration of the body 22.

In a deformed state in which it can be used as an interpositional ophthalmological implant between the sclera and the uveal tissue the body 22 of the implant 20 has a curvature in a direction perpendicular to a plane defined by the two dimensions of the implant that are perpendicular to the thickness. This curvature is preferably concave to adapt optimally to the anatomy of the patient.

The body of the implant may be made of a material that has a Young's modulus between 30 and 60 kg/cm² inclusive. In this case the body of the implant may comprise at least one material that is chosen from the following materials: PTFE, polysiloxane, hydrophilic or hydrophobic acrylate hydrogels.

Alternatively the body of the implant made be made of a material that has a Young's modulus between 30,000 and 2,500,000 kg/cm². In this case the body of the implant may comprise at least one material that is chosen from the following materials: polypropylene, polymethylmethacrylate, titanium, stainless steel, Nitinol.

The body of the implant may have properties of releasing one or more substances. Such substances are for example anti-infection substances and/or anti-inflammation substances. Thus these may be antibiotic substances and/or cortisone substances or anti-cortisone substances.

The body 22 of the implant 20 may be entirely or locally covered with one or more substances or have a judiciously chosen surface state so that the cellular growth of the surrounding tissues onto the implant 20 is inhibited or limited by the substance or substances or the surface state.

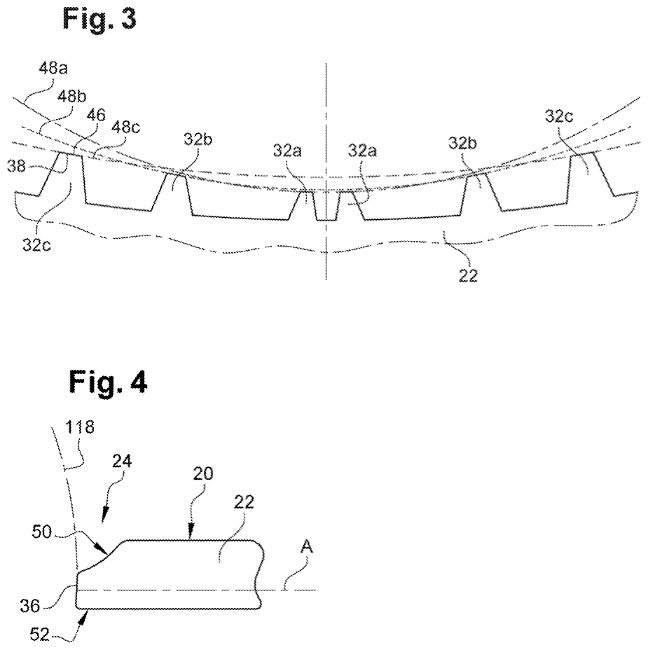

An example of an anterior edge 24 comprising groups of protuberances extending along circular trajectories having different radii of curvature is depicted in FIG. 3.

In this example the anterior edge 24 comprises three groups of protuberances 32: a first group of protuberances 32a, a second group of protuberances 32b and a third group of protuberances 32c. Three circular trajectories are represented: a first trajectory 48a, a second trajectory 48b and a third trajectory 48c. The radii of curvature of the trajectories increase from the first trajectory 48a to the third trajectory 48c. In other words the third trajectory 48c has a radius of curvature greater than the second trajectory 48b which has a radius of curvature greater than the first trajectory 48a.

The contact edge portions 38 of the first group of protuberances 32a extend along the first trajectory 48a. The contact edge portions 38 of the second group of protuberances 32b extend along the second trajectory 48b. The contact edge portions 38 of the third group of protuberances 32c extend along the third trajectory 48c.

In this FIG. 3 example the groups each comprise two protuberances 32. Generally speaking each of the groups of protuberances 32 may comprise at least one protuberance 32. Furthermore, the anterior edge 24 may comprise a plurality of groups of protuberances 32 respectively extending along a plurality of circular trajectories with different radii of curvature. It is therefore possible to produce an anterior edge capable of adapting to a plurality of or even an infinite number of different anatomies of the root of the ciliary muscle.

Referring to FIG. 4, the thickness of the anterior edge 24 may be reduced relative to the thickness of the body 22. This thinning may equally be produced on said at least one protuberance 32.

This thinning of the anterior edge 24 is preferably produced on the end portion of the anterior edge 24, which makes it possible to reduce the area of the anterior edge 24 bearing on the root of the ciliary muscle 118 for a given thickness of the body 22. This makes it possible to free up space between the root of the ciliary muscle 118 and the body 22 of the implant 20, thereby improving the collection and circulation of aqueous humor toward the body of the implant.

The anterior edge 24 comprises an upper portion 50 and a lower portion 52. The thickness of the anterior edge is therefore reduced at the level of the upper portion 50. The contact edge 36 is carried by the lower portion 52 of the anterior edge 24.

This thinning of the anterior edge 24 preferably extends from the contact edge 36 to the body 22 along the longitudinal axis A over a distance less than or equal to 1.5 mm.

The thinning is preferably effected over the entirety of the length of the anterior edge 24. Alternatively, this thinning is produced over only a portion of the length of the anterior edge 24.

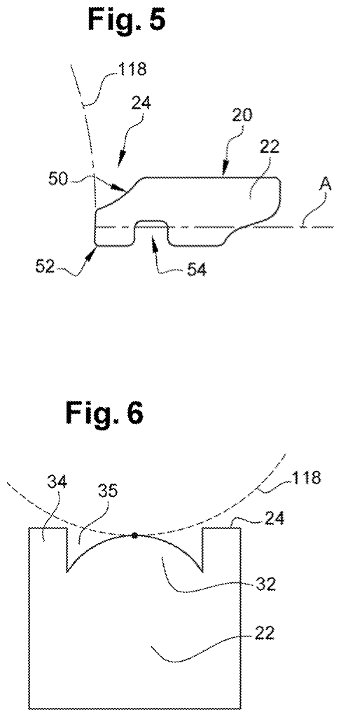

Referring to FIG. 5, the anterior edge 24 may equally comprise a local thinning 54 of the thickness. This local thinning 54 is formed on the upper face or the lower face of the implant 20. This local thinning 54 is configured to favor the elastic deformation of the anterior edge 24, in particular of its distal part, making it possible to improve the area of contact between the root of the ciliary muscle 118 and the anterior edge 24. In fact, by virtue of the action of a force pressing the implant 20 onto the root of the ciliary muscle along the longitudinal axis A the local thinning 54 makes it possible to induce rotation of the distal part of the anterior edge 24 toward the local thinning 54. This deformation of the anterior edge 24 favors the contact between the anterior edge 24 and the root of the ciliary muscle 118.

The body of the implant may be pierced by orifices passing through its thickness. These orifices are for the passage of a flow of aqueous humor through the body 22, from one of the opposite large faces to the other opposite large face. The body may equally be pierced by a plurality of other orifices situated in other planes.

The body 22 of the implant 20 may comprise on at least one of its two opposite large faces a relief that is able to favor flow of the aqueous humor over said at least one large face.

The relief may for example take the form of channels or grooves on the opposite faces. The channels or grooves may be formed on the surface or through the body 22. These channels or grooves are preferably arranged substantially parallel to a direction that extends from the anterior edge 24 of the body 22 to the posterior edge 26.

The relief may take the form of recesses on said at least one large face or a roughness conferred thereon.

It will be noted that the body 22 of the implant 20 can combine through-orifices, channels/grooves and recesses.

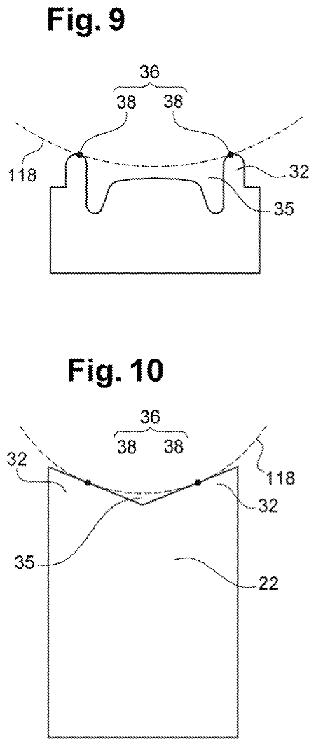

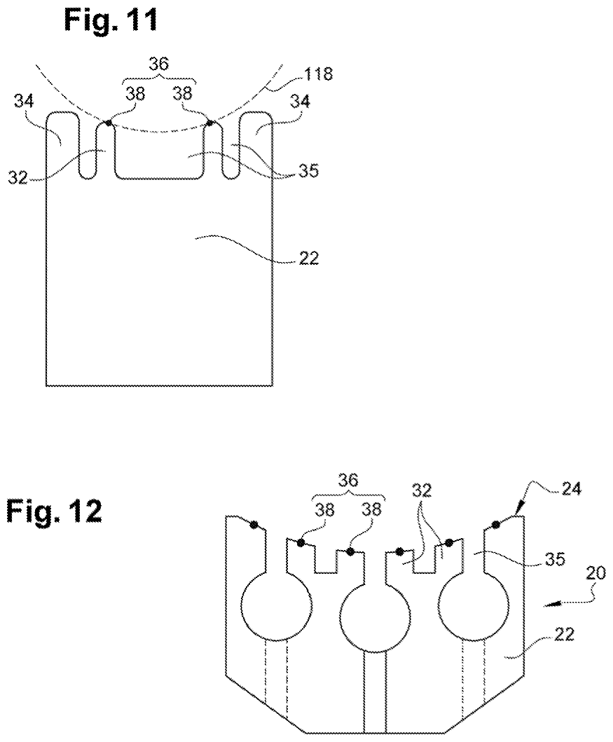

FIGS. 6 to 14 represent embodiments of the body 22 of the implant 20 and most importantly of the anterior edge 24.

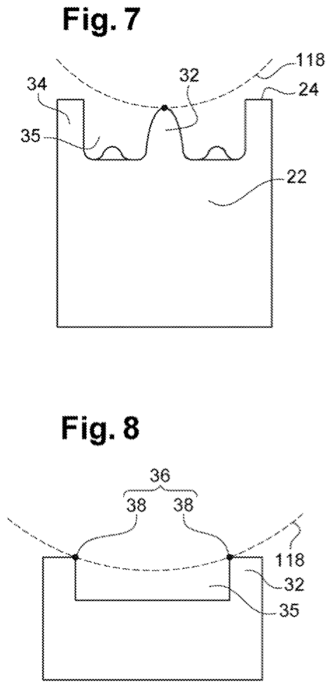

FIGS. 6 and 7 represent examples of anterior edges 24 with different geometries comprising a single protuberance 32 enabling local contact. The zone of local contact between the implant 20 and the root of the ciliary muscle is symbolized in the figures by a dot. As mentioned hereinabove the zone of local contact may be localized or linear.

In FIG. 6 the anterior edge 24 comprises a protuberance 32 formed by a curved central portion extending between two retaining arms 34. These retaining arms 34 conduct the aqueous humor to favor flow thereof toward the body 22 of the implant in such a manner as to increase the efficacy of aqueous humor collection.

More generally, the anterior edge may comprise at least one retaining arm 34 projecting from the body toward the exterior thereof. These retaining arms 34 are not configured to come into contact with the root of the ciliary muscle 118. The objective of these retaining arms 34 is to conduct the aqueous humor to favor the flow thereof toward the body of the implant 22. The anterior edge 24 preferably comprises at least two retaining arms 34, disposed for example at the lateral edges of the anterior edge 24.

In one particular configuration a protuberance 32 on the anterior edge 24 may have a retaining arm function, in particular when the anterior edge 24 comprises at least two protuberances 32.

Collection recesses or spaces 35 are formed between the protuberance 32 and the retaining arms 34.

In FIG. 7 the protuberance 32 is an elongate protuberance. The protuberance 32 also extends in the middle of two retaining arms 34. Collection recesses 35 are also formed between the retaining arms 34 and the protuberance 32.

The FIGS. 6 and 7 examples of the geometry of the anterior edge 24 comprise retaining arms 34. Alternatively the anterior edge 24 may be devoid of retaining arms 34.

FIGS. 8 to 11 represent examples of anterior edges 24 with different geometries comprising two protuberances 32 enabling local contact at the level of two zones of the root of the ciliary muscle 118. The zones of local contact between the implant 20 and the root of the ciliary muscle 118 are symbolized in these figures by a dot. As mentioned hereinabove the zones of local contact may be localized or linear.

In FIGS. 8 to 11 the two protuberances 32 are formed at the level of a peripheral portion of this anterior edge 24. In other words the two protuberances 32 are formed at the level of the lateral extremities of the anterior edge 24. A collecting recess 35 is formed between the two protuberances 32.

In all these examples of anterior edges 24 in FIGS. 8 to 11 the contact profile 44 of the contact edge 36 extends along a concave trajectory.

In FIGS. 8, 9 and 11 the two protuberances are elongate protuberances. Here these two protuberances 32 form arms for retaining aqueous humor in the collecting recess 35.

The protuberances 32 in FIG. 10 are each formed by a rectilinear and inclined portion of the anterior edge 24. The rectilinear and inclined portions meet at the level of a central portion of the anterior edge 24 to form a collecting recess 35.

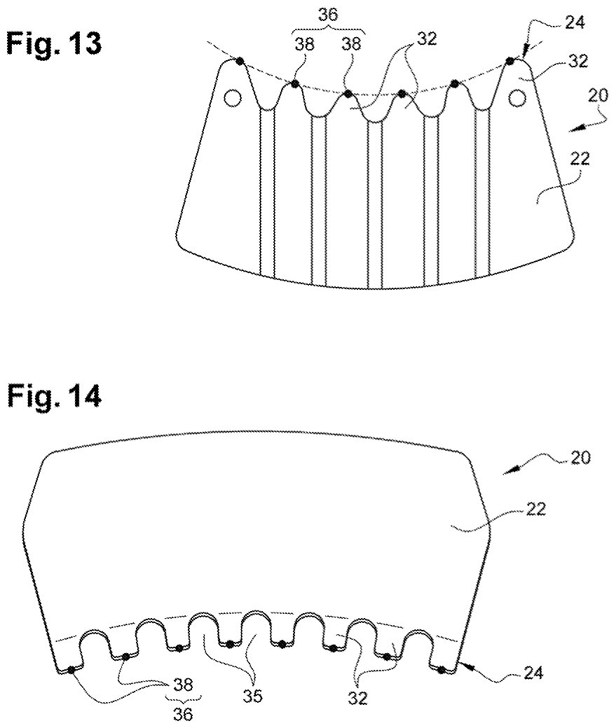

FIGS. 12 and 13 represent examples of anterior edges 24 with different geometries comprising a plurality of protuberances 32 enabling local contact at the level of a plurality of zones of the root of the ciliary muscle 118. The zones of local contact between the implant 20 and the root of the ciliary muscle 118 are symbolized in these figures by a dot.

The contact profile 44 of the contact edge 36 extends along a concave trajectory in FIGS. 12 and 13.

In FIGS. 12 and 13 the protuberances 32 form contact edge portions 38 all aligned on the same trajectory, in particular the same circular trajectory.

In FIG. 12 the protuberances 32 form linear contact edge portions 38 in such a manner as to obtain linear zones of contact with the root of the ciliary muscle 118.

Collecting recesses 35 are formed between the protuberances 32. Collecting channels or grooves are formed between these collecting recesses 35 and the posterior edge 26. These collecting recesses 35 have a circular or pseudo-circular shape.

In FIG. 13 the protuberances 32 form curved contact edge portions 38 in such a manner as to obtain localized zones of contact with the root of the ciliary muscle 118.

Collecting orifices are formed at the lateral extremities of the anterior edge 24. Generally speaking the collecting orifices may be formed all along the anterior edge 24. Collecting channels or grooves extend from the anterior edge 24 toward the posterior edge 26 to enable a flow of aqueous humor.

FIG. 14 depicts an example of an anterior edge 24 the contact profile 44 of which extends along a plurality of circular trajectories having different radii of curvature.

In a similar manner to FIG. 3 the anterior edge in FIG. 14 comprises a plurality of groups of protuberances 32 of which each of the contact edge portions 38 extends along a different trajectory. In particular the FIG. 14 example comprises four pairs of protuberances 32 extending along four circular trajectories with different radii of curvature.

In order to increase the flow of aqueous humor a passage may be created through the root of the ciliary muscle to establish direct communication between the anterior chamber of an eye and its supraciliary space (cyclodialysis). Said passage is obtained by an incision made during the surgery to implant the implant. However, lacking additional precautions, this opening will be rapidly closed up because of natural scarring. It may be made more permanent if a non-resorbable and non-deformable object holds it open.

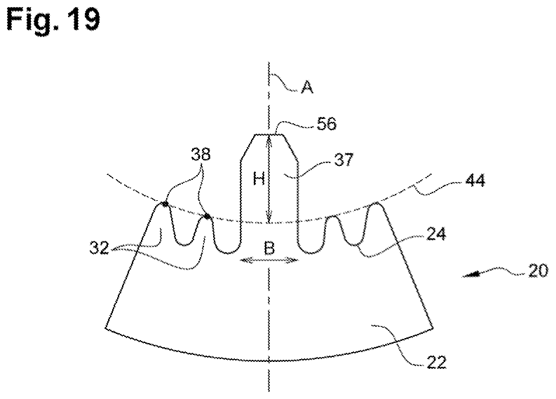

To this end FIG. 19 shows an embodiment of the invention in which the anterior edge 24 comprises a local extension projecting out of the body 20 as seen in a view in projection in the plane containing the width and the length. This local extension 37, called a needle, differs from a protuberance as defined above in that it extends beyond the contact profile 44 and is intended to pass completely through the root of the ciliary muscle to enable a long-lasting opening to be established between the anterior chamber and the supra-ciliary space.

The anterior edge 24 may include one or more needles 37.

Said at least one needle 37 extends along a longitudinal axis A passing through the distal point 56.

Said at least one needle 37 may be of elongate shape. As seen in a view in projection in the plane containing the width and the length said needle 37 may have a transverse dimension B in a direction perpendicular to the longitudinal axis A less than its longitudinal dimension H.

Alternatively, said at least one needle 37 may be of flattened shape, i.e., as seen in a view in projection in the plane containing the width and the length, have a transverse dimension B in a direction perpendicular to the longitudinal axis A greater than its longitudinal dimension H.

The lateral walls of said at least one needle 37 may be rectilinear, curved or a combination of rectilinear and curved walls.

Said at least one needle 37 may also include variations of width, i.e. of transverse dimension B, or a geometry making it possible to facilitate the passage of said at least one needle 37 through the ciliary muscle. A geometry of this kind must be complemented by a distal shape appropriately chosen to prevent the withdrawal of said needle from the anterior chamber, in particular thanks to widening of the distal point 56 of said at least one needle relative to its base, and/or thickening of said distal point.

It has been determined that the thickness of the root of the ciliary muscle varies from 200 to 500 microns depending on the person. In a preferred embodiment of the invention said at least one needle 37 has a longitudinal dimension H between 0.2 and 2.5 mm inclusive.

In another preferred embodiment said at least one needle 37 has a transverse dimension B between 0.2 and 2.5 mm inclusive.

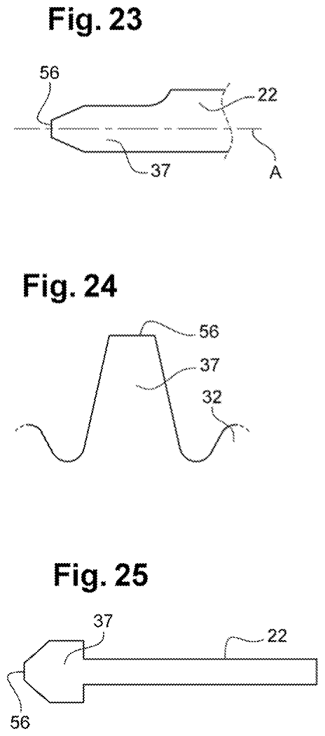

Referring to FIG. 23, the thickness of the distal point 56 of said at least one needle may be thinned relative to the thickness of the body 22, thus constituting an element facilitating passing it through the ciliary muscle.

Referring to FIG. 25, the thickness of the distal point 56 of said at least one needle may be increased relative to the thickness of the body 22, thus constituting a device for resisting withdrawal of said at least one needle from the anterior chamber.

The distal point 56 of said at least one needle 37 may include a thinning followed by a thickening to facilitate passing it through the ciliary muscle and then to resist its withdrawal.

The thinning and the thickening may be effected in the plane perpendicular to the thickness and/or in the plane of thickness of the implant 20.

Said at least one needle 37 may be pierced by orifices passing through it longitudinally or contain grooves or recesses. These orifices, grooves or recesses enable the passage of a flow of aqueous humor through the ciliary muscle.

Said at least one needle may be positioned either on the lateral edge of the implant 20 or between two protuberances 32.

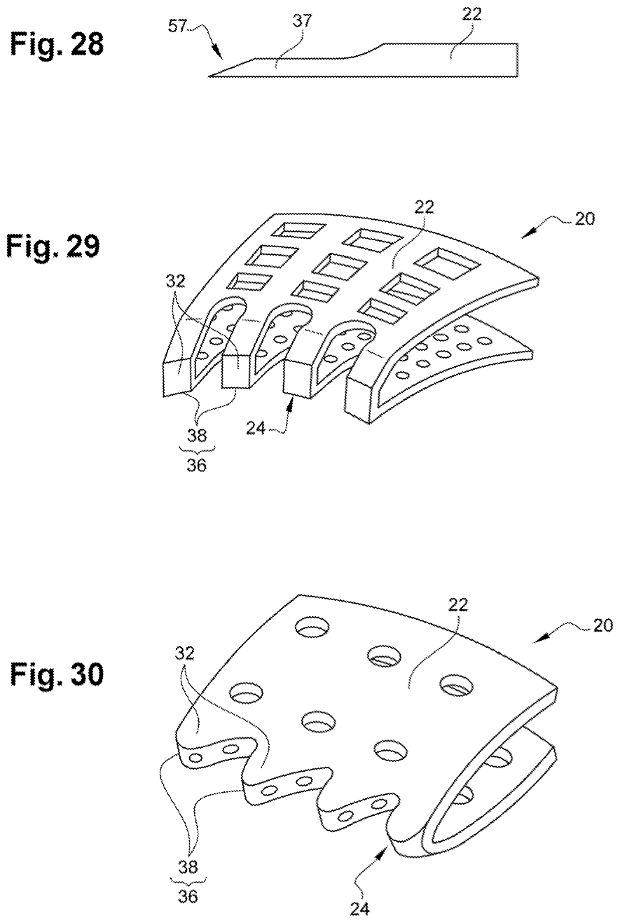

Said at least one needle 37 may have a perforation distal point 57 configured to perforate a wall of the root of the ciliary muscle without previously forming an opening in that wall. In this case the needle 37 is auto-perforating because it enables insertion through tissues without previous opening. The perforation capacity of the perforation distal point 57 may be obtained by one or more beveled edges at the level of the distal end of the needle so that the perforation distal point 57 has a triangular or truncated triangular section in a plane perpendicular to the thickness of the implant 20. FIG. 28 depicts an example of a needle including a perforation distal point 57.

FIG. 19 depicts an example of an implant 20 including four protuberances 32 and a needle 37 the longitudinal distance H of which is greater than the transverse distance B.

FIG. 22 depicts an example of an implant 20 including four protuberances 32 and a needle 37 the longitudinal distance H of which is less than the transverse distance B. The needle further includes internal channels and grooves.

FIG. 24 depicts an example of the geometry of said at least one needle enabling facilitated insertion through the ciliary muscle.

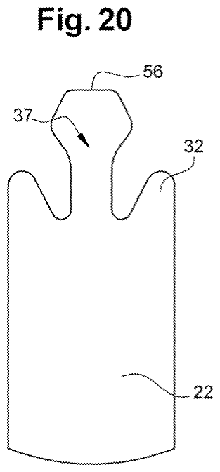

In FIG. 20 the distal point of the at least one needle 37 has a first example of a geometry enabling facilitated insertion through the ciliary muscle and resistance to withdrawal.

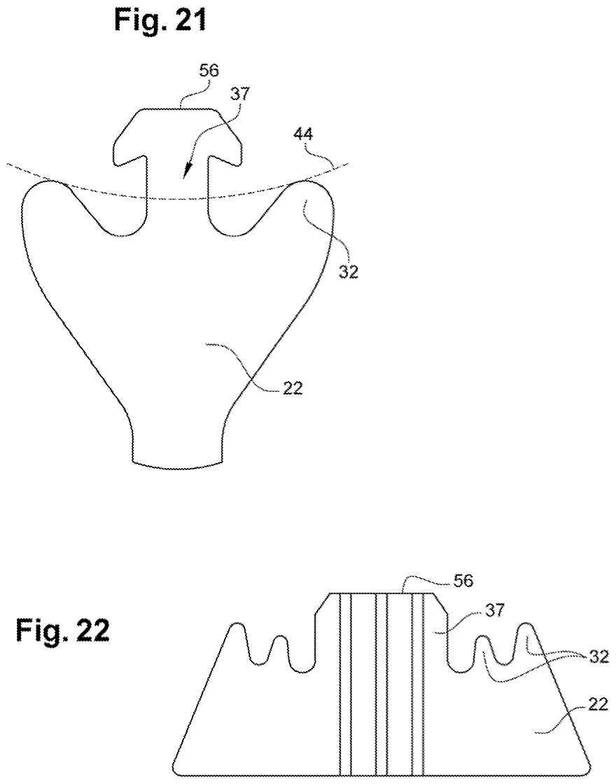

In FIG. 21 the distal point of the at least one needle 37 has a second example of a geometry enabling facilitated insertion through the ciliary muscle and resistance to withdrawal.

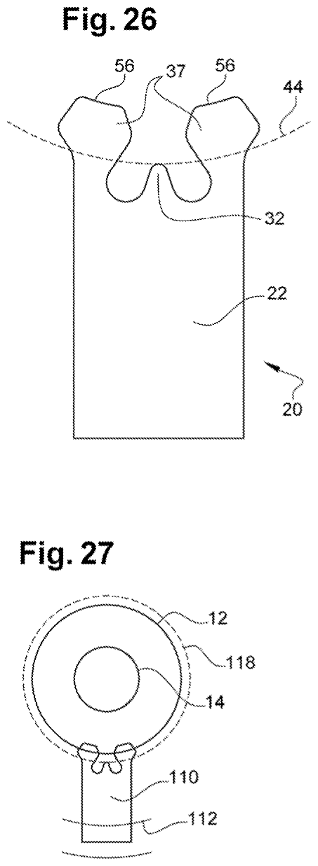

FIG. 26 shows an implant 20 with two needles 37 and one protuberance 32.

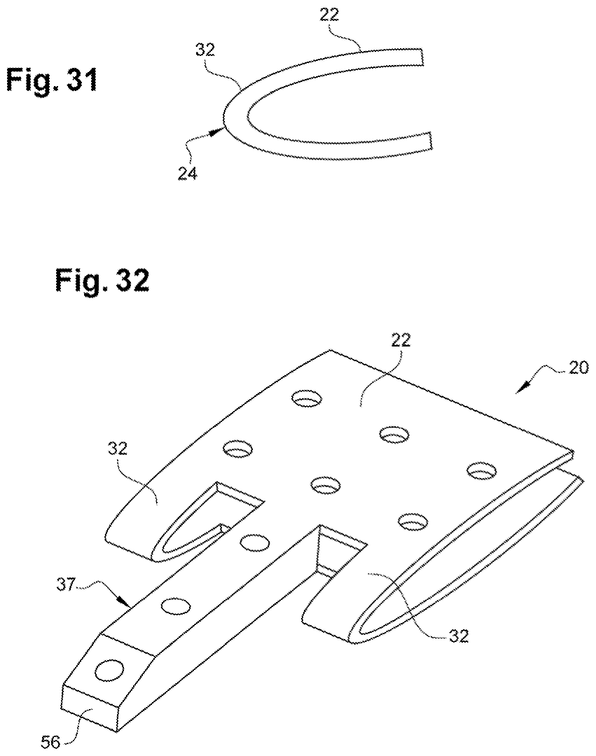

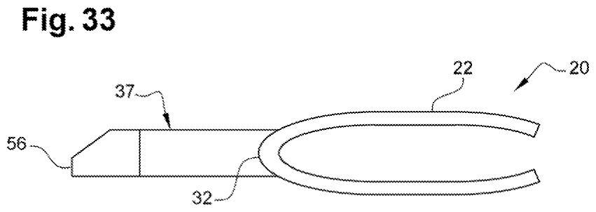

FIGS. 29 to 33 represent embodiments of the implant 20 in the form of a hollow body 22. Thus a central cavity is formed between the upper and lower faces of the body 22. The upper and lower faces are connected to one another at the level of one of the edges of the body 22, preferably the anterior edge 24. The body 22 may be obtained by means of one plate folded on itself. The junction between the upper and lower faces at the level of the anterior edge 24 also forms at least one protuberance 32.

A needle 37 may equally be formed on the anterior edge 24. This needle 37 is preferably also hollow. The needle 37 comprises at least two walls, an upper wall and a lower wall.

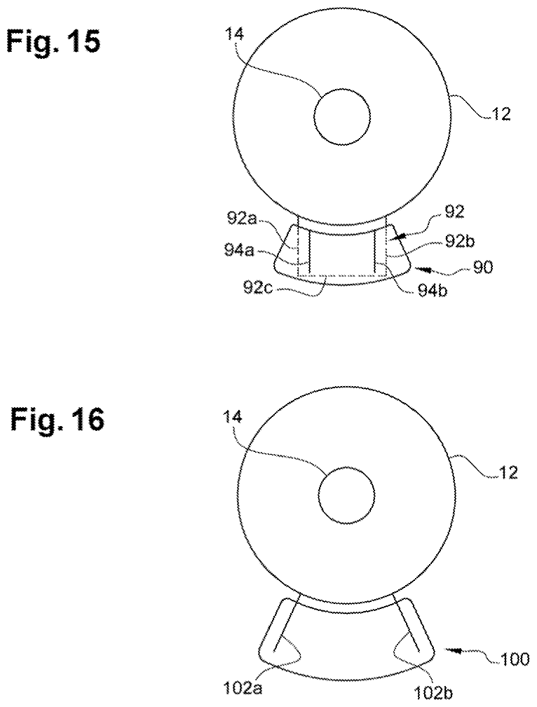

FIG. 15 depicts the implantation of an implant 90 using a first implantation method in accordance with one embodiment of the invention. The implant 90 is intentionally represented in a simplified manner but conforms to the invention described hereinabove. This first method is used to complement a conventional anti-glaucoma surgical intervention or any intra-ocular intervention when lowering the intra-ocular pressure is desirable. The trabeculectomy and the sclerectomy necessitate cutting one or more scleral flaps of the eye that will be raised in order to continue the intervention. A scleral flap 92 (depicted in dotted line in FIG. 15) is obtained by incising the sclera in one or two planes and to a variable depth, on three sides: two substantially parallel incisions 92a, 92b that extend from the cornea 12 and away from the latter and a third incision 92c perpendicular to the other two incisions and at a distance from the cornea. The resulting cutting with three incisions forms one or more scleral flaps. After raising the scleral flap or flaps two incisions (94a, 94b in FIG. 15) are made extending as far as the ciliary body, inside the flap 92, in order to be able to slid the implant between the deep scleral plane and the ciliary body. The incisions are spaced from one another by at least 2 mm for example.

In a variant that is not represented a single incision is made in the scleral plane to achieve the same objective.

The implantation method used may then comprise a step of introducing a visco-elastic substance, for example of the hyaluronic acid type, through at least one of the incisions made, between the sclera and the ciliary body, in order to separate these two tissues previously back-to-back. This will enable implantation of the implant without trauma to the overlying and underlying structures.

This step is effected using an injection instrument such as an injection cannula having a diameter of the order of 20 to 30 g.

A small quantity of substance, for example 0.05 mm³ is injected.

The surgeon forms the same number of openings between the anterior chamber and the supraciliary space as the number of needles 37 that the implant 20 includes. These openings are formed at the location of said needles.

If the surgeon so wishes supplementary openings may be made between the anterior chamber and the supraciliary space.

This opening or these openings may be produced ab-interno or ab-externo, for example by mechanical action (scalpel, spatula, . . . ) or by laser. As for the supplementary openings, they may be made at the time of surgery or subsequently.

The implantation method also comprises a step of introducing an instrument such as forceps with foam edges through one of the two incisions 94a, 94b that extend in the depthwise direction as far as the ciliary body. The forceps exit via the incision and grasp the implant to place it between the sclera and the ciliary body.

During a subsequent step using a micro-surgical instrument such as a spatula with foam edges the implant 90 is positioned as close as possible to the trabecula (in a manner concentric with the limbus), as described above, in order to collect the maximum aqueous humor.

Another type of instrument or device such as an injector may be used enabling implantation and deployment of the implant and positioning thereof in the space between the sclera and the ciliary body (supra-choroidal space).

During another step the scleral flap or flaps are folded down and optionally sutured.

The first method described applies also to implanting a plurality of implants according to the invention. Generally speaking, at least one different incision (or even two in the FIG. 15 example) is made for implanting each different implant.

FIG. 16 depicts the implantation of an implant 100 using a second implantation method in accordance with an embodiment of the invention. The implant 100 is intentionally represented in a simplified manner but conforms to the invention described hereinabove.

This method is very similar to the first method except that the second method does not complement a conventional intervention but may constitute an intervention in itself.

In this method:
two incisions 102a, 102b that are preferably radial relative to the cornea or preferably parallel to one another (like the incisions 94a, 94b in FIG. 15) are made, the anterior extremity of the incisions being situated between 0 and 3 mm posterior to the limbus (the zone of transition between the cornea and the sclera), with a length from 1 to 4 mm for example, and continued as far as the ciliary body,
a visco-elastic substance, for example of the hyaluronic acid type, may be injected via one of the two incisions,
if necessary, depending on the requirement or the intention of the surgeon, openings between the anterior chamber and supra-ciliary space are made by means of the steps described for the first method,
the sclera is raised in order to enable the insertion and the positioning of the implant 100. The insertion steps are identical to those described above for the first method,
the final step of suturing the incisions is again optional.

In a variant embodiment that is not represented a single incision is made in this second method and suffices for implanting an implant interpositionally between the sclera and the ciliary body.

FIG. 27 depicts the implantation of an implant 110 using a third implantation method according to one embodiment of the invention. The implant 110 is intentionally represented in a simplified manner but conforms to the invention described hereinabove.

The second method described also applies to implanting a plurality of implants in accordance with the invention. Generally speaking, at least one different incision (or even two in the FIG. 27 example) is to be made for implanting each different implant.

This method may be carried out to complement ordinary filtration surgery or independently of any other interventional action.

In this method an incision 112 that is preferably concentric with the limbus is made between 1 and 5 mm from the limbus over a distance of 0.5 to 4 mm for example.

A visco-elastic substance, for example of hyaluronic acid type, may be injected through this incision.

The surgeon makes a number of openings between the anterior chamber and the supra-ciliary space equal to the number of needles 37 that the implant 20 includes. These openings are at the location of said needles. The surgeon may wish to make supplementary openings between the anterior chamber and the supra-ciliary space.

This opening or these openings may be produced ab-interno or ab-externo, for example by mechanical action (scalpel, spatula, . . . ) or by laser. As for the supplementary openings, they may be made at the time of surgery or subsequently.

The implant is then inserted with the aid of forceps under the sclera along an axis perpendicular to the limbus, in the direction of the anterior chamber until the protuberances of the implant contact the root of the ciliary muscle 118. For implants 110 including one or more needles 37 this implies that the needles pass through the openings made by the surgeon and are then present in the interior chamber.

Another type of instrument or device such as an injector enabling implantation, deployment of the implant and positioning thereof in the space situated between the sclera and the ciliary body (supra-choroidal space) may be used.

If the whole of the implant is not inserted under the sclera the remaining posterior part of the implant is tucked under the sclera in the direction away from the anterior chamber.

The final step of suturing the incisions is again optional.

In the embodiment in which the needle 37 includes a perforation distal point 57 the surgeon may dispense with making one or more incisions between the anterior chamber and the supra-ciliary space as described for the aforementioned three methods. In this case the step of positioning the implant 20 against the root of the ciliary muscle includes perforation by the perforation distal point 57 of the root of the ciliary muscle to insert the needle 37 into the anterior chamber until said at least one protuberance 32 is in contact with the root of the ciliary muscle.

It will be noted that the implants 90, 100 and 110 represented in FIGS. 15, 16 and 27 may be any of the implants described hereinabove. The methods of implanting implants described hereinabove apply to any implant according to the invention and in particular to a permanent interpositional ophthalmological implant between the sclera and the uveal tissue that comprises a uveo-compatible thin body formed in one piece, the body of the implant comprising two opposite edges that are spaced from one another in one of the two directions perpendicular to the thickness.

The implant may further include any one (or more than one, or even all) of the features described in the general description and in the various embodiments and variants.

The third method described also applies to implanting a plurality of implants according to the invention. Generally speaking, at least one different incision is to be made for implanting each different implant.

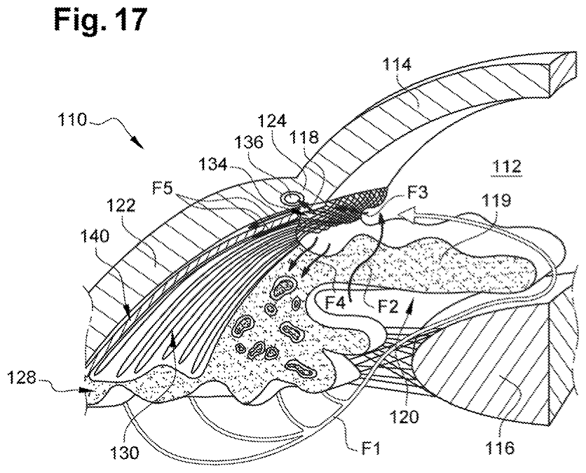

FIG. 17 represents in section an implant according to one embodiment of the invention that has been implanted using one of the methods described hereinabove.

This section of a part of an eye 110 represents the anterior chamber 112 that is disposed between the cornea 114 and the crystalline lens 116 delimited in its peripheral part by the iris 119.

The posterior chamber 120 is disposed behind the iris 119.

The sclera 122 is connected to the periphery of the cornea 114 via the limbus 124 (the zone of the change of radius of curvature between the sclera and the cornea). The sclera 122 covers the ciliary body 128, which is connected to the iris 119 and comprises the ciliary muscle 130 on which the sclera 122 bears.

The trabecula 134 disposed between the cornea and the iris serves as a filter and has the aqueous humor that circulates in the anterior chamber 112 pass through it.

Schlemm's canal 136 is situated between the sclera and the cornea behind the trabecula 134.

The various arrows F1, F2, F3 and F4 depict the paths or trajectories taken by the aqueous humor:
F1 represents the conventional path or flow taken by the aqueous humor entering the anterior chamber 112;
F2 represents the diffusion path or flow taken by the aqueous humor to enter the anterior chamber 112;
F3 represents the conventional path or flow taken by the aqueous humor leaving the anterior chamber 112 via the trabecula 134 and directed toward Schlemm's canal 136;
F4 represents the conventional uveo-scleral physiological flow of the aqueous humor leaving the anterior chamber 112.

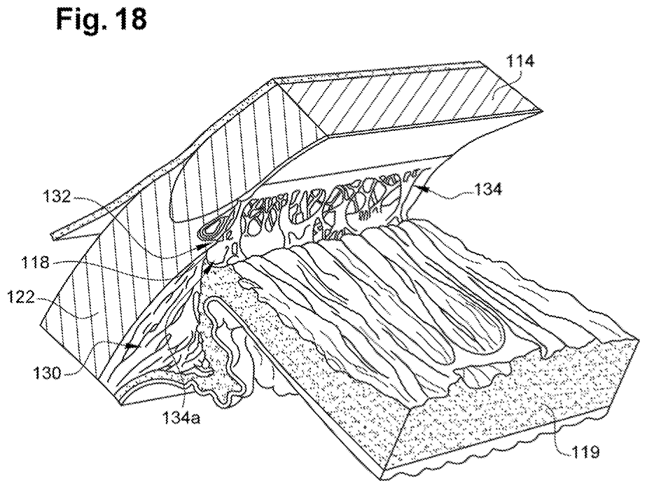

An implant 140 according to one embodiment of the invention has been disposed between the sclera 122 and the ciliary muscle 130 as described hereinabove. This implant is positioned as close as possible to the root of the ciliary muscle 118 (via its concave anterior edge) in order to exert its permanent spacing effect at the most appropriate place while respecting the insertion of ciliary muscle 130 in the scleral spur. FIG. 18 is a more detailed view to a larger scale of the structure of the irido-corneal corner without the implant. As represented in this figure, the scleral spur 132 in which the ciliary muscle 130 is inserted is situated above the posterior part 134a of the trabecula 134.

The spacing produced at this location between the sclera and the ciliary body enables permanent collection of the aqueous humor as close as possible to the zone of the uveo-scleral physiological flow (the spacing effect creates a zone of lower resistance to the flow of the aqueous humor). An implant of this kind positioned in this way achieves a significant improvement in terms of increasing the uveo-scleral physiological flow.

The uveo-scleral physiological flow is increased by a supplementary flow fraction through the posterior part of the trabecula (ciliary trabecula), as represented in FIG. 17 by the arrows F5 above and below the implant. The flow of this supplementary fraction is obtained thanks to the spacing effect of the implant between the sclera and the ciliary body, as close as possible to the root of the ciliary muscle 118, without damaging the latter, however.

1. An interpositional ophthalmological implant for permanent disposition between the sclera and uveal tissue, comprising:
a uveo-compatible body, the body having three dimensions in space, namely a length and a width that are perpendicular to one another and a thickness, the body of the implant comprising a first or anterior edge intended to be oriented in the direction of the anterior chamber of an eye in contact with the root of the ciliary muscle and a second or posterior edge opposite the anterior edge relative to the body, the anterior edge having, as seen in a view in projection in the plane containing the width and the length, at least one protuberance oriented toward the exterior of the body to enable at least one local contact of said at least one protuberance on the root of the ciliary muscle.

2. The interpositional ophthalmological implant as claimed in claim 1, in which the anterior edge comprises a plurality of protuberances oriented toward the exterior of the body, said protuberances conjointly forming a discontinuous or local contact edge on the root of the ciliary muscle.

3. The interpositional ophthalmological implant as claimed in claim 2, in which the contact edge defines a contact profile of the anterior edge on the root of the ciliary muscle, said contact profile extending along a concave trajectory.

4. The interpositional ophthalmological implant as claimed in claim 3, in which the contact profile of the contact edge extends along at least one circular trajectory.

5. The interpositional ophthalmological implant as claimed in claim 3, in which the contact profile of the contact edge comprises at least one profile portion extending along a circular trajectory.

6. The interpositional ophthalmological implant as claimed in claim 5, in which the contact profile of the contact edge comprises at least two profile portions extending along circular trajectories having different radii of curvature.

7. The interpositional ophthalmological implant as claimed in claim 6, in which the contact profile of the contact edge comprises at least one first profile portion extending along a first circular trajectory having a first radius of curvature and at least one second profile portion extending along a second circular trajectory having a second radius of curvature greater than the first radius of curvature to enable the contact edge to come into contact with the root of the ciliary muscle having a plurality of possible anatomical radii.

8. The interpositional ophthalmological implant as claimed in claim 7, in which the first profile portion is formed at the level of a central zone of the contact edge, the second profile portion being disposed at the level of a peripheral zone of the contact edge.

9. The interpositional ophthalmological implant as claimed in claim 7, in which the second profile portion is disposed on either side of the first profile portion along the contact edge.

10. The interpositional ophthalmological implant as claimed in claim 4, in which at least one radius of curvature of the circular trajectory is greater than or equal to 4.5 mm and less than or equal to 6 mm.

11. The interpositional ophthalmological implant as claimed in claim 4, in which at least one radius of curvature of the circular trajectory is greater than 6 mm and less than or equal to 7.5 mm.

12. The interpositional ophthalmological implant as claimed in the preceding claim 1, in which said at least one protuberance is configured to allow at least one localized contact on the root of the ciliary muscle.

13. The interpositional ophthalmological implant as claimed in claim 1, in which said at least one protuberance is configured to enable at least one linear contact on the root of the ciliary muscle.

14. The interpositional ophthalmological implant as claimed in claim 12, wherein the contact edge defines a contact profile of the anterior edge on the root of the ciliary muscle, said contact profile extending along a concave trajectory, and
in which each protuberance comprises a contact edge portion, each of said contact edge ports extending along the concave trajectory.

15. The interpositional ophthalmological implant as claimed in claim 1, in which the thickness of the anterior edge is thinned relative to the thickness of the body.

16. The interpositional ophthalmological implant as claimed in claim 15, in which the anterior edge comprises an upper portion and a lower portion, the thickness of the anterior edge being thinned at the level of the upper portion.

17. The interpositional ophthalmological implant as claimed in claim 15, in which the body comprises a local thinning of thickness situated set back from the anterior edge and configured to enable elastic deformation of the anterior edge by the action of a force tending to press the anterior edge against a bearing surface, for example the root of the ciliary muscle.

18. The interpositional ophthalmological implant as claimed in claim 1, in which the anterior edge comprises at least one needle extending toward the exterior of the body and projecting beyond said at least one protuberance in such a manner as to be able to pass through a wall of the root of the ciliary muscle when said at least one protuberance is in contact with the root of the ciliary muscle.

19. The interpositional ophthalmological implant as claimed in claim 18, in which the needle comprises a perforation distal point configured to perforate a wall of the root of the ciliary muscle without an opening being formed in said wall beforehand.

20. The interpositional ophthalmological implant as claimed in claim 18, in which said at least one needle comprises an aqueous humor collecting channel extending from one end of said at least one needle to the body of the implant.

21. The interpositional ophthalmological implant as claimed in claim 1, in which the body forms at least one aqueous humor collecting recess opening at the level of the anterior edge.

22. The interpositional ophthalmological implant as claimed in claim 21, wherein the anterior edge comprises a plurality of protuberances oriented toward the exterior of the body, said protuberances conjointly forming a discontinuous or local contact edge on the root of the ciliary muscle, and
in which said at least one recess is formed between two protuberances.

23. The interpositional ophthalmological implant as claimed in claim 1, in which the body of the implant is elastically deformable in such a manner as to be able to be folded without permanent deformation in order to be manipulated by a micro-instrument or injected using an ophthalmological injection system.

24. The interpositional ophthalmological implant as claimed in claim 23, wherein at least one radius of curvature of the circular trajectory is greater than or equal to 4.5 mm and less than or equal to 6 mm, and
in which the body of the implant is configured to have at least one radius of curvature of the circular trajectory that is greater than or equal to 4.5 mm and less than or equal to 6 mm when the implant is disposed in a deformed state corresponding to a disposition of the body of the implant between the sclera and uveal tissue.

25. The interpositional ophthalmological implant as claimed in claim 23, wherein at least one radius of curvature of the circular trajectory is greater than 6 mm and less than or equal to 7.5 mm, and
in which the body of the implant is configured to have at least one radius of curvature of the circular trajectory that is greater than 6 mm and less than or equal to 7.5 mm when the implant is disposed in said deformed state corresponding to a disposition of the body of the implant between the sclera and the uveal tissue.

26. Interpositional ophthalmological implant as claimed in claim 23, in which the body of the implant has in a deformed state in which it can be used as an interpositional ophthalmological implant between the sclera and the uveal tissue a concave curvature in a direction perpendicular to a plane defined by the two dimensions of the implant that are perpendicular to the thickness.

27. The interpositional ophthalmological implant as claimed in claim 1, in which the body of the implant is made of a material that has a Young's modulus between 30 and 60 kg/cm² or between 30,000 and 2,500,000 kg/cm².

28. The interpositional ophthalmological implant as claimed in claim 1 in which the body of the implant is not elastically deformable and has a permanent curvature in a direction perpendicular to a plane defined by the two dimensions of the implant that are perpendicular to the thickness.

29. The interpositional ophthalmological implant as claimed in claim 1, in which the body of the implant comprises at least one material that is chosen from the following materials: PTFE, polysiloxane, hydrophilic or hydrophobic acrylate hydrogels.

30. The interpositional ophthalmological implant as claimed in claim 1, in which the body of the implant has two opposite large faces separated from one another by the thickness of the body.

31. The interpositional ophthalmological implant as claimed in claim 30, wherein the body of the implant is not elastically deformable and has a permanent curvature in a direction perpendicular to a plane defined by the two dimensions of the implant that are perpendicular to the thickness, and
in which the two opposite large faces comprise an upper face and a lower face separated by the thickness, the body of the implant having one of the following conformations in a direction perpendicular to a plane defined by the two dimensions of the implant that are perpendicular to the thickness:
the upper face is plane and the lower face is concave,
the upper face is plane and the lower face is convex,
the upper and lower faces are convex,
the upper and lower faces are plane.

32. The interpositional ophthalmological implant as claimed in claim 30, in which the body of the implant is pierced by orifices passing through its thickness and/or the body of the implant comprises on at least one of its two opposite large faces a relief that is adapted to favor flow of the aqueous humor across said at least one large face.

33. The interpositional ophthalmological implant as claimed in claim 32, in which the relief on said at least one large face of the body takes the form of recesses formed on said at least one large face or a roughness conferred thereon.

34. The interpositional ophthalmological implant as claimed in claim 1, in which the thickness of the body of the implant is between 50 and 1000 μm.

35. The interpositional ophthalmological implant as claimed in claim 1, in which the body of the implant has properties of releasing one or more substances.

36. The interpositional ophthalmological implant as claimed claim 1, in which the posterior edge is at a distance of less than 1.0 mm from the anterior edge.
